(19) United States
(12) Patent Application Publication
(10) Pub. No.: US 2025/0161048 A1
(43) Pub. Date: May 22, 2025

(54) Annuloplasty Ring For Receiving A Replacement Valve

(71) Applicants: Northwestern University, Evanston, IL (US); Cardiac Valve Innovations, LLC, Winnetka, IL (US)

(72) Inventors: Patrick M. McCarthy, Winnetka, IL (US); Hieu Cong Nguyen, Denver, CO (US); Connor Wright Rowley, Littleton, CO (US)

(73) Assignees: Northwestern University, Evanston, IL (US); Cardiac Valve Innovations, LLC, Winnetka, IL (US)

(21) Appl. No.: 18/948,268

(22) Filed: Nov. 14, 2024

Related U.S. Application Data
(60) Provisional application No. 63/600,528, filed on Nov. 17, 2023.

(51) Int. Cl. A61F 2/24

(52) U.S. Cl. CPC A61F 2/2445; A61F 2210/0009; A61F 2210/0057; A61F 2230/0095; A61F 2250/0018; A61F 2250/0037

(57) ABSTRACT

An annuloplasty ring includes an elastic elongated core having first regions and second regions, wherein the first regions are more stretchable than the second regions and are adapted for positioning at the commissures of a target native heart valve. The first regions and the second regions may enable the annuloplasty ring to both re-establish coaptation of the leaflets of a diseased native heart valve and establish a seal around a prosthetic replacement heart to prevent paravalvular leakage after a “Valve In Ring Procedure”.

### RELATED APPLICATIONS

This application claims priority to and benefit of U.S. Provisional Application Ser. No. 63/600,528, filed Nov. 17, 2023, and entitled ANNULOPLASTY RING FOR RECEIVING A REPLACEMENT VALVE, which is hereby incorporated herein by reference in its entirety.

### FIELD OF THE INVENTION

The present invention relates to treatments for reestablishing coaptation of a native heart valve to prevent regurgitant flow, and further preventing paravalvular leakage around a replacement heart valve, such as including, but not limited to, a mitral valve, tricuspid valve, aortic valve, or pulmonic valve.

### BACKGROUND OF THE INVENTION

Annuloplasty rings have been significant in the advancement of treating diseased heart valves and have been known in the art for several years. In use, annuloplasty rings are sutured, clipped, or otherwise secured around a perimeter of a heart valve annulus or opening to help reduce the diameter of an overly enlarged (e.g., diseased) heart valve. This reduction in diameter helps compress the heart valve and allows its leaflets to reestablish coaptation to, in turn, reduce or eliminate regurgitant flow through the heart valve.

However, in some patients, while an implanted annuloplasty ring may reestablish coaptation of a native heart valve to properly treat regurgitation, the deterioration of the valve may continue over time to a point where complete valve replacement is necessary. Historically, complete valve replacements have required open-heart surgeries which involve the excision of the annuloplasty ring followed by the subsequent implantation of a replacement heart valve. Although this multi-step process has proved useful and has saved and prolonged the lives of numerous patients, the excision and implantation process is both time consuming and challenging for the operating physician. This may make such a procedure potentially dangerous for a patient. For example, if the condition of the diseased heart valve and the surrounding tissue is damaged, the excision of the previously implanted annuloplasty ring can make it very difficult for the operating physician to find suitable healthy tissue for attaching a prosthetic heart valve.

Generally, the necessity of removing a previously implanted annuloplasty ring arises from the rigid, and/or non-conformable nature of most existing annuloplasty rings. This may contribute to the annuloplasty ring constricting a native heart valve and/or preventing a prosthetic replacement valve to be placed within an implanted annuloplasty ring. A rigid annuloplasty ring may also, in some patients, distort the prosthetic replacement valve and result in valve regurgitation.

Additionally, the shape and size of some existing annuloplasty rings may not correspond sufficiently well to the prosthetic replacement valve to prevent a regurgitant leak between the annuloplasty ring and the prosthetic valve (e.g., paravalvular regurgitation) from immediately or eventually arising. As an example, many annuloplasty rings are roughly “D shaped” to correspond to the shape of a native heart valve annulus, but prosthetic replacement valves are generally circular or round. As such, the spatial relationships between existing annuloplasty rings and prosthetic valves have often prohibited, or complicated, the placement of a prosthetic valve within a previously implanted annuloplasty ring.

### SUMMARY

Disclosed herein are systems, devices, and/or methods relating to an annuloplasty ring molded from an elastic material.

In some examples, the techniques described herein relate to an annuloplasty ring for receiving a replacement heart valve, the annuloplasty ring including: an elongated core including a first region and a second region, wherein the first region is more stretchable than the second region and is adapted for positioning at commissures of a target native heart valve, the first region being made solely from an elastic material; a cover encompassing the elongated core; wherein the elongated core is stretchable from a resting state to a stretched state to receive the replacement heart valve; and wherein the first region is adapted to impart a squeezing force on the replacement heart valve so as to prevent paravalvular leakage.

In some examples, the techniques described herein relate to an annuloplasty ring, wherein the second region includes a first stiffener adapted to help maintain a shape of a posterior area of the annuloplasty ring.

In some examples, the techniques described herein relate to an annuloplasty ring, wherein the first stiffener includes a wire including a pair of end loops.

In some examples, the techniques described herein relate to an annuloplasty ring, wherein the first stiffener extends between about 110 degrees and about 130 degrees around an aperture of the annuloplasty ring.

In some examples, the techniques described herein relate to an annuloplasty ring, wherein the second region includes a second stiffener adapted to help maintain a shape of an anterior area of the annuloplasty ring.

In some examples, the techniques described herein relate to an annuloplasty ring, wherein the first stiffener or the second stiffener defines a vertical curvature to cause the annuloplasty ring to form a three-dimensional saddle shape.

In some examples, the techniques described herein relate to an annuloplasty ring, wherein the first stiffener defines a length that is longer than a length defined by the second stiffener.

In some examples, the techniques described herein relate to an annuloplasty ring, wherein the elongated core forms a complete loop.

In some examples, the techniques described herein relate to an annuloplasty ring for receiving a replacement heart valve, the annuloplasty ring including: an elongated core including a pair of first regions and a pair of second regions, wherein the pair of first regions are adapted for positioning at commissures of a target native heart valve are more stretchable than the pair of second regions, and wherein the pair of first regions are included solely of a silicone-based material; a cover encompassing the elongated core; wherein the elongated core is stretchable from a resting state to a stretched state to receive the replacement heart valve; and wherein the pair of first segments are configured to impart a squeezing force to the replacement heart valve so as to prevent paravalvular leakage.

In some examples, the techniques described herein relate to an annuloplasty ring, wherein the pair of second regions are separated from each other by the pair of first regions.

In some examples, the techniques described herein relate to an annuloplasty ring, wherein the pair of second regions and the pair of first regions collectively form a complete loop.

In some examples, the techniques described herein relate to an annuloplasty ring, wherein one of the pair of second regions includes a first stiffener adapted to help maintain a shape of a posterior area of the annuloplasty ring; and wherein another of the pair of second regions includes a second stiffener adapted to help maintain the shape of an anterior area of the annuloplasty ring.

In some examples, the techniques described herein relate to an annuloplasty ring, wherein the first stiffener and the second stiffener are curved to cause the annuloplasty ring to form a three-dimensional saddle shape.

In some examples, the techniques described herein relate to an annuloplasty ring, wherein the first stiffener and the second stiffener are each included of a polished wire defining a pair of end loops.

In some examples, the techniques described herein relate to an annuloplasty ring, wherein a ratio between a maximum width of an aperture defined by the elongated core and a maximum length of the aperture is about 4:3, and wherein the maximum width is measured between the pair of second regions the maximum length is measured between the pair of first regions.

In some examples, the techniques described herein relate to a method of treating a diseased heart valve, the method including: implanting an annuloplasty ring around a heart valve to cause leaflets of the heart valve to coapt, wherein the annuloplasty ring includes a pair of first regions made solely from a silicone-based material, and wherein pair of first regions are more stretchable than the pair of second regions to allow the annuloplasty ring to be stretched from a resting state to a stretched state upon each opening of the heart valve; monitoring the heart valve periodically for an extended period; and installing a prosthetic valve within said annuloplasty ring if the heart valve relapses into a diseased state.

In some examples, the techniques described herein relate to a method, wherein implanting the annuloplasty ring around the diseased heart valve includes positioning each of the pair of first regions at a commissure of the heart valve where the pair of leaflets meet.

In some examples, the techniques described herein relate to a method, wherein implanting the annuloplasty ring around the heart valve includes positioning each of the pair of second regions along a leaflet of the heart valve.

In some examples, the techniques described herein relate to a method, wherein positioning each of the pair of second regions along a leaflet of the heart valve includes: resisting, by a first stiffener of the pair of second regions, stretching of a posterior area of the annuloplasty ring; and resisting, by a second stiffener of the pair of second regions, stretching of an anterior area of the annuloplasty ring.

In some examples, the techniques described herein relate to a method, wherein the method first includes selecting the annuloplasty ring from a plurality of annuloplasty rings based on a maximum width of an aperture of the annuloplasty ring and a maximum length, and wherein a ratio between the maximum width and the maximum length is about 4:3.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1 is a perspective view of an annuloplasty ring with an elongated core, in accordance with at least one example.

FIG. 2 is a perspective view of the annuloplasty ring of FIG. 1 with a fabric covering, accordance with at least one example.

FIG. 3 is a perspective view of a first stiffener of an annuloplasty ring, in accordance with at least one example.

FIG. 4 is a perspective view of a second stiffener of an annuloplasty ring, in accordance with at least one example.

FIG. 5 is a side view of an annuloplasty ring, in accordance with at least one example.

FIG. 6 is a perspective view of an annuloplasty ring including a plurality of core segments, in accordance with at least one example.

FIG. 7 is a perspective view of an annuloplasty ring, in accordance with at least one example.

FIG. 8 is a top view of an annuloplasty ring, in accordance with at least one example.

FIG. 9 is a cross-section of an annuloplasty ring, in accordance with at least one example.

FIG. 10 is a perspective view of an annuloplasty ring implanted around a valve annulus, in accordance with at least one example.

FIG. 11 is a perspective view of an annuloplasty ring attached to a ring holder, in accordance with at least one example.

### DETAILED DESCRIPTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

In view of the above background above, there remains a distinct need for an improved annuloplasty ring which can successfully treat a diseased native heart valve (e.g., resolve valve regurgitation), and later, provide a platform that is suitable to receive and retain a prosthetic valve in the event the native valve deteriorates further. As may be appreciated, such an annuloplasty ring would not require excision from the patient, and thus would reduce both difficulty of the operation for an operating physician and the risk to the patient.

Disclosed herein are devices and methods relating to an annuloplasty ring including a highly elastic core material adapted to improve stress handling characteristics over existing annuloplasty rings. For example, the annuloplasty ring disclosed herein may possess a greatly increased yield limit when compared to conventional semi-rigid annuloplasty rings which may become susceptible to developing fractures or permanently deforming under increased stress. Moreover, the annuloplasty ring disclosed herein may be impervious to repetitive stress fatigue caused by the cyclic loading and unloading of annuloplasty rings during the diastole and systole cycle of a beating heart. In addition to improving durability and longevity, the annuloplasty ring disclosed herein may also help to more effectively seal around a cylindrical prosthetic replacement valve by elastically conforming to an entire circumference of a prosthetic valve.

In some examples, the annuloplasty ring disclosed herein may include regions of increased elasticity configured for placement at or near commissures of a native heart valve. In such regions, the annuloplasty ring may be constructed solely from an elastic material without any rigid or semi-rigid bracing features. Such a configuration may enable the annuloplasty ring to maximize stretchability at trigone locations associated with the least amount of radial movement, thus the amount lowest radial force, to improve physiological function of both a native valve and/or a subsequently implanted prosthetic valve by allowing for less restriction or constriction about the valve's annulus.

The annuloplasty ring disclosed herein may also include at least one region of increased stiffness. For example, the annuloplasty ring disclosed herein may include strategically embedded stiffeners only in areas subject to the highest radial forces to help ensure that annuloplasty ring will completely seal against a prosthetic valve to thereby prevent paravalvular leakage, without significantly decreasing elasticity in locations where maximum stretchability is desirable. In view of all the above, the annuloplasty ring disclosed herein may provide benefits to both patients and operating physicians.

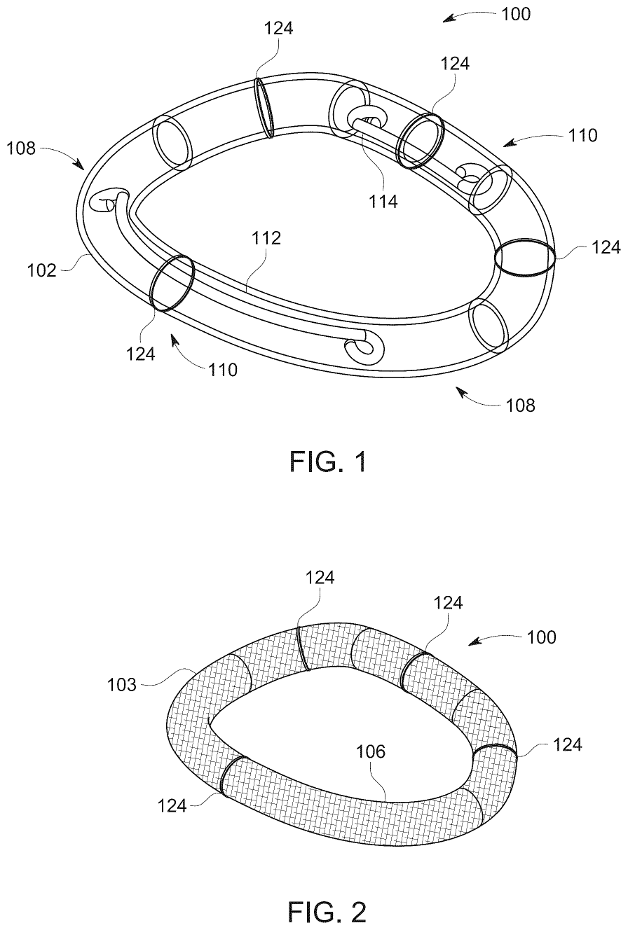

FIG. 1 is a perspective view of an annuloplasty ring 100 including an elongated core 102. The elongated core 102 is generally shaped to, when implanted, impart a squeezing (e.g., a radial inward) force around an enlarged native heart valve's annulus to restore coaptation of its valve leaflets. For example, the elongated core 102 may form a 3-dimensional saddle shape that corresponds to the mitral valve or the tricuspid valve, or a planar and circular shape that corresponds to the aortic valve or the pulmonic valve. Additionally, the elongated core 102 is adapted to stretch from a resting state to a stretched state to receive a prosthetic replacement heart valve during a subsequently performed “Valve-In Ring” implantation procedure. Details of how various annuloplasty rings may be implanted to re-establish leaflet coaptation and receive replacement valves are disclosed in U.S. Pat. No.: 11,337,808, which is hereby incorporated by reference in its entirety.

Generally, the elongated core 102 may form a complete loop shape which allows the annuloplasty ring 100 to be implanted around the annulus of a diseased native valve without significantly impacting natural opening and closing movements, such as by elastically restraining the native valve's annulus only to the extent necessary to re-establish coaptation of an enlarged valve's leaflet. Additionally, such an elastic material may enable the elongated core 102 to, at later time, elastically stretch like a rubber band from a resting state to a stretched state to seal around a prosthetic valve inserted into an aperture 106 of the annuloplasty ring 100 to replace the native valve. As may be appreciated, this may allow a prosthetic valve to be implanted without excision of the annuloplasty ring 100.

In use, the elongated core 102 is encompassed by a fabric covering 103 as shown in FIG. 2. Thus, in FIG. 1, the elongated core 102 is illustrated with the fabric covering 103 removed to reveal various other features of the elongated core 102. The fabric covering 103 may be representative of a double velour fabric, a Dacron fabric, a braided polyester fabric, or any other covering that is used in the art to promote the in-growth of tissue and help anchor sutures.

The elongated core 102 of the annuloplasty ring 100 may be collectively comprised of first regions 108 and second regions 110. The first regions 108 may be representative of various segments, sections, or portions of the elongated core 102 constructed entirely from an elastic (e.g., silicone-based) material. In one such example, such the material may be NuSil MED-4840 mixed with about 20% weight per weight with barium sulfate (“BaSO4”). As may be appreciated, the presence of barium sulfate may enable an operating physician to visualize the entire circumference of the elongated core 102 during fluoroscopic imaging. In other examples, the silicone-based material may be made from pure (e.g., 100%), or substantially pure, silicone. As such, the first regions 108 are areas which exhibit highly elastic, rubber band-like properties.

For this reason, the first regions 108 may be configured to align with the commissures, such as medial and lateral commissures, and/or the trigone, of a diseased native heart valve when the annuloplasty ring 100 is positioned about the valve's annulus. As one skilled in the art will recognize, the commissures and/or the trigone are subject to the least amount of radial movement and thus experience lowest radial forces during normal opening and closing of a valve. Thus, the elastic, flexible nature of the first regions 108 may help to improve the performance of a native valve over existing annuloplasty rings by allowing for more natural movements of the native valve while still supporting the diseased valve's annulus to maintain coaptation and thereby correct regurgitant flow.

The high level of elasticity of the first regions 108 may also allow the annuloplasty ring 100 to be squeezed and/or folded into a small space as may be necessary during a robotic and/or port access implantation. Moreover, the high level of flexibility of the first regions 108 may eliminate the need to perform the extensive and complex fatigue analysis required for the safe use of the variable length metallic features employed in some annuloplasty rings, such as springs, coils, or other stretchable or flexible metallic ring elements.

The second regions 110 may be representative of various segments, sections, or portions of the elongated core 102 which possess increased rigidity or resiliency relative to the first regions 108. For this reason, the second regions 110 may be shaped and sized to align with areas of a native heart valve that are located between its commissures. As one skilled in the art will recognize, these areas are subject to the greatest radial movement and thus experience the highest radial forces during opening and closing of a valve.

Generally, the second regions 110 are made from the same highly elastic material that forms each of the first regions 108, however, each of the second regions 110 may be stiffer than each of the first regions 108. In some examples, the increased stiffness of the second regions 110 may be accomplished via the inclusion of an embedded bracing feature, such as a first stiffener 112 and/or a second stiffener 114. For example, the first stiffener 112 may be strategically placed to help one of the second regions 110 maintain the shape (e.g., resist or limit radial distortion) of a posterior area of the elongated core 102, and the second stiffener 114 may be strategically placed, and spaced circumferentially apart from the first stiffener 112, to help maintain the shape (e.g., resistance or limit radial distortion) of an anterior area of the elongated core 102.

Additionally, in some examples, the increased stiffness of the second regions 110 may be at least partially accomplished by increasing the cross-sectional diameter of the elongated core 102 within each of the second regions 110, relative to the cross-sectional diameter of the elongated core 102 within each of the first regions 108. Alternatively, the reduced stiffness and/or increased flexibility of the first regions 108 may be at least partially accomplished by reducing the cross-sectional diameter of the elongated core 102 within each of first regions 108, relative to the cross-sectional diameter of the elongated core 102 with each of the second regions 110.

As may be appreciated, such strategic placement of the second regions 110 may help to prevent undesirable overstretching of the elastic elongated core 102 between a pair of valve commissures, such as near the middle of leaflet, that could lead to regurgitant flow through a native mitral valve, or a paravalvular leak between the annuloplasty ring 100 and a prosthetic valve placed therein. In still further examples, the elongated core 102 may alternatively be adapted for a native valve that includes three leaflets and three commissures, such as the tricuspid valve, the aortic valve, or the pulmonary valve. In such examples, it may be appreciated that an additional stiffener with another of the second regions 110 may be spaced circumferentially apart from the first stiffener 112 and the second stiffener 114, and each of the three stiffeners may extend between separate first regions 108 each located at a different commissure. Thus, the annuloplasty ring 100 may be alternatively adapted to prevent overstretching of the elongated core 102 between a trifecta of valve commissures.

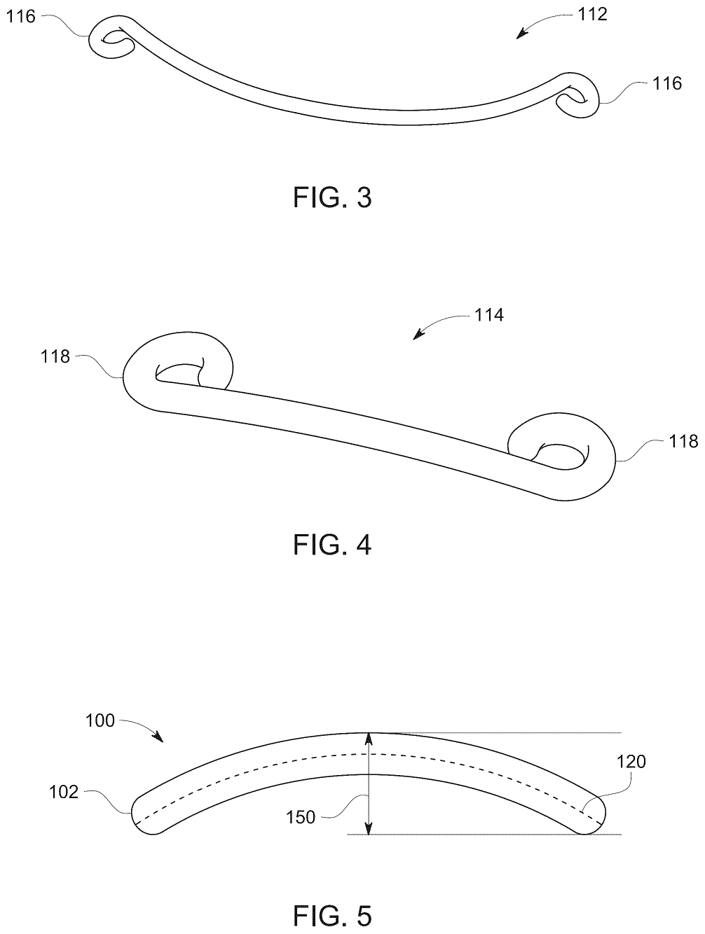

In addition to limiting stress induced radial distortion of the elongated core 102, the first stiffener 112 and the second stiffener 114 may also be configured to help impart a three-dimensional shape to the elongated core 102. In some such examples, the first stiffener 112 may possess a curvature with respect to, or within, at least two orthogonally oriented planes. For example, the first stiffener 112 may define both a lateral curvature, such as shown in the top view of FIG. 3, and a vertical curvature, such as shown by line 120 in the side view of FIG. 5). In some examples, only the second stiffener 114 may define a vertical curvature, such as shown by the line 120, which may force the elongated core 102 to form a “saddle” shape. In further examples, both the first stiffener 112 and the second stiffener 114 may define similar, or different, vertical curvatures with respect to each other and/or the line 120 to help the elongated core 102 correspond to the target annulus and form a three-dimensional shape. While not shown in FIG. 4, the second stiffener 114 may, in still further examples, also define a lateral curvature that may be similar or different to a lateral curvature defined by the first stiffener 112.

As may be appreciated, in such examples of the annuloplasty ring 100, the elongated core 102 may also define a height 150 (FIG. 5). The height 150 may be a maximum vertical distance between the top of the annuloplasty ring 100 and a flat surface on which the annuloplasty ring 100 may be positioned. In some such examples, the height 150 may measure between about, but not limited to, 5 millimeters and about 12 millimeters. In one specific example, the height 150 may measure about 6.5 millimeters. In another specific example, the height 150 may measure about 11 millimeters.

In other examples, the first stiffener 112 may form only a lateral curvature, and the second stiffener 114 may not be curved at all, such as to help the annuloplasty ring 100 correspond to a valve annulus that forms a planar “O” shape to conform to a generally circular valve annulus, such as of an aortic valve or pulmonic valve. In still further examples, the first stiffener 112 and/or the second stiffener 114 may be configured to form uniform or compound lateral and/or vertical curvatures that help the elongated core 102 correspond to various other valves. In view of all the above, it is to be appreciated that the number of individual first regions 108 and individual second regions 110, and their placement (e.g., the relative angular lengths or sizes) about the circumference of the aperture 106 may vary widely according to the intended implantation location and orientation of the annuloplasty ring 100.

In some examples, the first stiffener 112 and the second stiffener 114 may each be representative of, for example, but not limited to, lengths or segments of biocompatible wire. In one example, the first stiffener 112 or the second stiffener 114 may be made from MP35N Alloy Wire stock. Such wire stock is composed of Carbon (about 0.025% max), Manganese (about 0.15% max), Phosphorus (about 0.015% max), Sulfur (about 0.01% max), Silicon (about 0.15% max), Chromium (about 19.00-21.00%), Nickel (about 33.00-37.00%), Molybdenum (about 9.00-10.50%), Cobalt (about 29.15-36.65%), Titanium (about 1.00% max), and Iron (about 1.00% max). In further examples, the first stiffener 112 and the second stiffener 114 may be constructed of other biocompatible materials including, but not limited to, Nitinol, stainless steel, cobalt chromium, titanium, nickel, or various alloys.

In some examples, the first stiffener 112 and the second stiffener 114 may possess a yield strength of between about, but not limited to, 135 KSI and about 155 KSI. In one example, the first stiffener 112 and the second stiffener 114 may possess a yield strength of about 145 KSI. In some examples, the first stiffener 112 and the second stiffener 114 may possess an ultimate yield strength of between about, but not limited to, 245 KSI and about 265 KSI. In one example, the first stiffener 112 and the second stiffener 114 may possess an ultimate yield strength of about 255 KSI. In some examples, the first stiffener 112 and the second stiffener 114 may exhibit an elongation at yield of about 2 percent to about 4 percent. In some examples, the first stiffener 112 and the second stiffener 114 may be polished to impart a smooth finish that may help reduce the risk of damage, fracture, or breakage of the first stiffener 112 or the second stiffener 114.

In some examples, the first stiffener 112 may include a first pair of end loops 116 and the second stiffener 114 may include a second pair of end loops 118. The first pair of end loops 116 and the second pair of end loops 118 are configured to significantly increase the cross-sectional area of opposite end regions or end portions of the first stiffener 112 and the second stiffener 114, respectively.

The first pair of end loops 116 and the second pair of end loops 118 may help to anchor the first stiffener 112 and the second stiffener 114 within the elongated core 102. For example, the first pair of end loops 116 and the second pair of end loops 118 may help to resist movement or migration within the elongated core 102 and prevent penetration through the elongated core 102 and/or fabric covering 103, which could otherwise be caused by aggressive handling or flexing of the annuloplasty ring 100, or during folding of the annuloplasty ring 100 during a port access implantation procedure. Further, the first pair of end loops 116 and the second pair of end loops 118 may help the first stiffener 112 and the second stiffener 114 retain their three-dimensional shapes (e.g., their curvatures). For example, one skilled in the art will recognize that the resilience of a wire segment may be increased by curling or curving the end of the wire segment in a direction opposite another curvature defined by the wire segment.

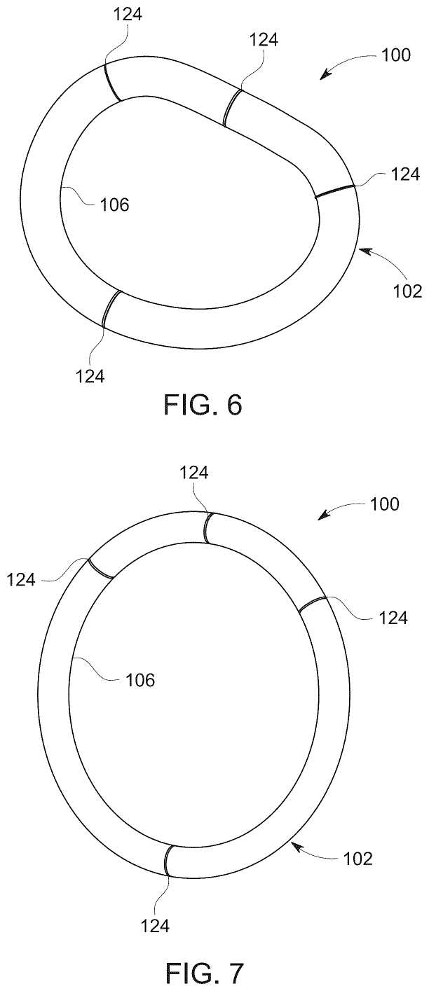

In some examples, the elongated core 102 may be manufactured from a plurality of core segments affixed to one another. In various examples, the plurality of core segments may include two, three, four, five, six, or other numbers of individual segments. In some examples, such as also shown in FIGS. 1-2 and 6-7, the annuloplasty ring 100 may include a plurality of marker sutures 124 adapted to help orient the elongated core 102 within a patient. In various examples, the plurality of marker sutures 124 may include two, three, four, five, six, or other numbers of individual marker sutures 124 respectively. In various examples, each of the plurality of marker sutures 124 may measure between about 0.1 millimeters and about 0.3 millimeters wide.

In some such examples, the relative sizes of the plurality of such core segments may be configured to ensure that the first pair of end loops 116 of the first stiffener 112 and the second pair of end loops 118 of the second stiffener 114 are spaced circumferentially apart from any junctions or connection points between the plurality of core segments. As may be appreciated, such a configuration may help to improve the overall durability of the elongated core 102 by keeping any junctions or connection points away from areas of the elongated core 102 that may experience increased internal stress.

From FIG. 7, it is also to be appreciated that the annuloplasty ring 100 may be implanted in the resting shape or state shown in FIGS. 1-2 and 6, which may be similar, but not limited to, to the “D” shape of a native mitral or tricuspid valve annulus. As one of skill in the art will recognize, as the silicone-based material of the elongated core 102 possesses no shape memory, the elongated core 102 may easily deform into a generally circular shape or stretched state, such as shown in FIG. 7, during replacement of the native valve (e.g., upon the insertion of a tubular or cylindrical prosthetic valve into the annuloplasty ring 100). As such, the annuloplasty ring 100 may also help to avoid paravalvular leakage that may be caused by permanent plastic deformation of some existing annuloplasty rings.

In some examples, the elongated core 102 of the annuloplasty ring 100 may alternatively be comprised of a single, integrally molded core section. In such an example, the elongated core 102 may otherwise be similar, or identical, to the annuloplasty ring 100 previously described above, such as by including the first regions 108, the second regions 110, the first stiffener 112, and the second stiffener 114, and the fabric covering 103. Additionally, while the elongated core 102 is shown in the above examples as defining a generally tubular or circular cross-sectional shape, it is to be appreciated that the elongated core 102 may also define other cross-sectional shapes such as, but not limited to, cuboidal, rectangular, triangular, heptagonal, or hexagonal cross-sectional shapes.

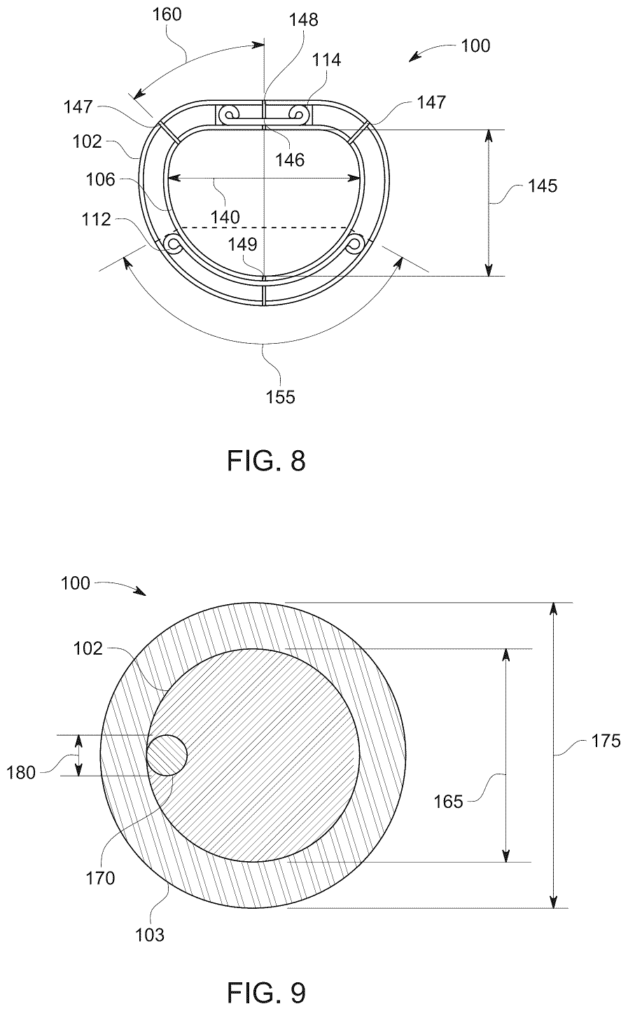

As shown in FIG. 8, the aperture 106 of the annuloplasty ring 100 may define a width 140 and a length 145. The width 140 may be the lateral distance across the aperture 106 of the annuloplasty ring 100, and the length 145 may be a longitudinal distance across the aperture 106 of the annuloplasty ring 100. Generally, the width 140 will, in use, correspond to a distance between two adjacent commissures (e.g., the commissure to commissure, “C-C”, or “top to bottom” distance) of a native valve, and the length 145 will correspond to an anteroposterior (“AP”, or “septal-lateral”) distance across the annulus of the native valve. In some examples, the width 140 may be between about 20 millimeters and about 45 millimeters, and the length 145 may be between about 14 millimeters and about 35 millimeters.

In some examples, the width 140 and the length 145 may be adapted to correspond to each other in a 4 to 3 ratio, respectively, such as to help establish a uniform sizing scheme for plurality of different rings produced for inventory. In some examples, such a plurality of different rings may include one, two, three, four, five, six, seven, eight, nine, ten, eleven, twelve, or more differently sized rings. In some examples, for a first ring size, the width 140 may be about 42 millimeters and the length 145 may be about 31 millimeters. In some examples, for a second ring size, the width 140 may be about 40 millimeters and the length 145 may be about 30 millimeters. In some examples, for a third ring size, the width 140 may be about 38 millimeters and the length 145 may be about 28 millimeters. In some examples, for a fourth ring size, the width 140 may be about 36 millimeters and the length 145 may be about 27 millimeters.

In some examples, for a fifth ring size, the width 140 may be about 34 millimeters and the length 145 may be about 25 millimeters. In some examples, for a sixth ring size, the width 140 may be about 32 millimeters and the length 145 may be about 24 millimeters. In some examples, for an eighth ring size, the width 140 may be about 30 millimeters and the length 145 may be about 22 millimeters. In some examples, for an eighth ring size, the width 140 may be about 28 millimeters and the length 145 may be about 21 millimeters. In some examples, for a ninth ring size, the width 140 may be about 26 millimeters and the length 145 may be about 19 millimeters. In some examples, for a tenth ring size, the width 140 may be about 24 millimeters and the length 145 may be about 18 millimeters.

The above configurations can help to improve the speed at which a physician is able to select an annuloplasty ring 100 for a particular patient (e.g., a ring precisely adapted to facilitate valve leaf coaptation and prevent valve systolic anterior motion). For example, by proportioning the lateral and longitudinal dimensions in a 4:3 ratio, an operating physician may be able to choose a correctly sized ring based on only one of a measured commissure to commissure (“C-C”) distance, or anteroposterior (“AP”) distance, of the patient.

In various examples, the first stiffener 112 may extend circumferentially within the annuloplasty ring 100 by various angular distances. For example, the first stiffener 112 may define a first angular distance 155 that measures between about, but not limited to, 110 and about 130 degrees. In one example, the first angular distance 155 may be about 120 degrees. Such a configuration has been found to effectively restrict undesirable annular dilation at a posterior section of a valve annulus without unduly limiting flexibility of the elongated core 102.

In some examples, the elongated core 102 may also define a second angular distance 160. The second angular distance 160 may be the circumferential distance, relative to the aperture 106, between a center point 146 of the second stiffener 114 and two adjacent marker sutures 147 of the plurality of marker sutures 124. In some examples, the second angular distance 160 may be between about 30 degrees and about 60 degrees. In one such example, the second angular distance 160 may be about 45 degrees. Additionally, in some examples such as shown in FIG. 9, the center point 146 may also coincide with an anterior marker suture 148 and may be axially aligned a posterior marker suture 149. The above configurations may be helpful in positioning the second stiffener 114 to effectively restrict undesirable annular dilation at the anterior section of a valve annulus.

As shown in FIG. 9, the elongated core 102 may define an outer diameter 165. The cross-section shown in FIG. 9 is taken through one of the second regions 110 of the annuloplasty ring 100 to reveal a stiffener 170, which may be representative of the first stiffener 112, the second stiffener 114, or any other stiffener embedded within the elongated core 102. In some examples, the outer diameter 165 may measure between about, but not limited to, 2 millimeters and about 3 millimeters. In one example, the outer diameter 165 may measure about 2.5 millimeters. In another example, the outer diameter 165 may measure about 2.25 millimeters or about 2.75 millimeters.

As further shown in FIG. 9, the fabric covering 103 may also define an outer diameter 175. In some examples, the outer diameter 175 may measure between about 5 millimeters and about 7 millimeters. In other words, the thickness of the fabric covering 103 may measure about, but not limited to, 3 millimeters and about 4 millimeters in thickness. In one example, the outer diameter 175 may be about 6 millimeters, representing a thickness of about 3.5 millimeters. In further examples, the outer diameter 175 may be about 5.5 millimeters or about 6.5 millimeters, representing a thickness of about 3 millimeters or 4 millimeters, respectively.

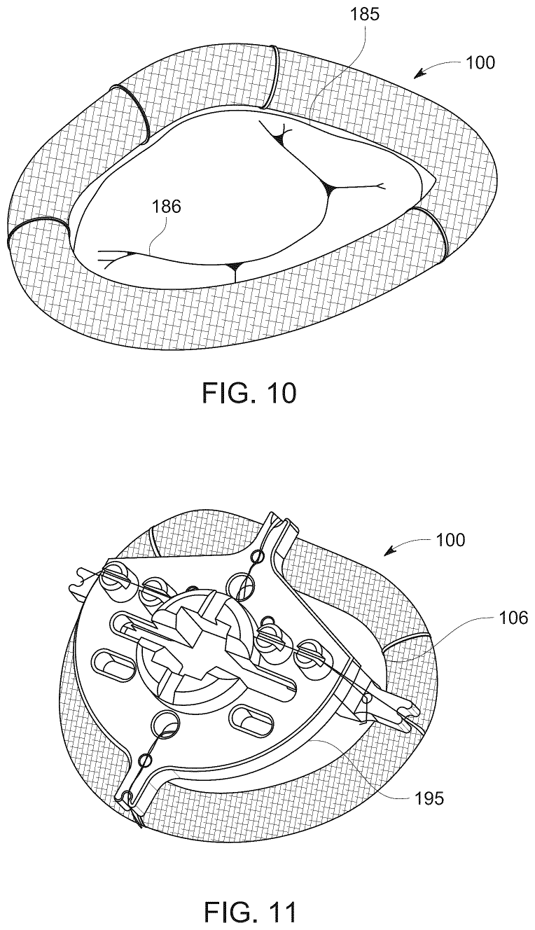

The stiffener 170 may also define an outer diameter 180. In some examples, the outer diameter 180 may measure between about 0.5 millimeters and about 1 millimeter. In one example, the stiffener 170 may define a diameter of about 0.71 millimeters. In further examples, the stiffener 170 may define a diameter of about 0.675 millimeters or about 0.725 millimeters. As shown in FIG. 10, the stiffener 170 may be biased within the elongated core 102 toward the aperture 106 (e.g., an inner circumference of the annuloplasty ring 100). In use, such a configuration may help to resist deformation of a surface of the elongated core 102 that is in contact with a prosthetic valve to help reduce the chance of a paravalvular leak there between, without affecting the high-level of stretchability of other portions of the elongated core 102.

Moreover, as one skill in the art will appreciate, by biasing the stiffener 170 toward the aperture 106, sutures may be looped around the annuloplasty ring 100, or may penetrate vertically through the elongated core 102, at any angular position (e.g., 360 degrees) about the annuloplasty ring 100 without being obstructed by the presence of the stiffener 170. As one of skill in the art will recognize, such a configuration may, relative to existing annuloplasty rings including variable length or stretchable portions, provide the operating physician with a greater number of suturing positions or enable the operating physician to use a greater number of sutures in securing the annuloplasty ring 100 to tissue.

As shown in FIG. 10, the annuloplasty ring 100 may, during a surgical impanation procedure, be positioned around a valve annulus 185 of a valve 186. In some examples, such an implantation procedure may begin with the measuring of the valve annulus 185 to obtain the commissure to commissure (“CC”) distance and/or the anteroposterior (“AP” or “septal-lateral”) distance. In one example, the operating physician may position a ring sizer at the valve annulus 185, as is generally known in the art. In one example, the ring sizer used may be the ring sizing system disclosed in U.S. Pat. No. 12,083,015, the entire contents of which is hereby incorporated by reference in its entirety.

Once the valve annulus 185 and its leaflets have been measured, the operating physician may select the annuloplasty ring 100 from an inventory (e.g., a plurality of different sized annuloplasty rings). In some examples, the operating physician may select the annuloplasty ring 100 based on a measured anteroposterior distance, to receive an annuloplasty ring 100 having a commissure-to-commissure distance that is about 133 percent greater than the measured anteroposterior distance. Alternatively, the operating physician may select the annuloplasty ring 100 based on a measured commissure to commissure distance, to receive an annuloplasty ring 100 having an anteroposterior distance that is about 33 percent less than the measured commissure to commissure distance.

Next, such as shown in FIG. 11, the selected annuloplasty ring 100 may be attached to ring holder 195. The ring holder 195 may be adapted to engage and stretch the annuloplasty ring 100 from within the aperture 106 to prevent relative movement there between. The ring holder 195 may, as well known in the art, be connected to a handle or other device that may vary in size, shape, and adjustability depending upon the intended surgical approach to the valve annulus 185. Subsequently, the operating physician may utilize the handle and the ring holder 195 to position the annuloplasty ring 100 at the valve annulus 185.

In some examples, positioning the annuloplasty ring 100 at the valve annulus 185 may include aligning the first regions 108 with commissures of the valve 186 where a plurality of (e.g., two or three) leaflets meet. In some examples, positioning the annuloplasty ring 100 at the valve annulus 185 may also include aligning the second regions 110 with, or positing the second regions 110 along, a plurality of (e.g., two or three) leaflets of the valve 186.

Once the annuloplasty ring 100 is located about the valve annulus 185, the annuloplasty ring 100 may be secured to the valve annulus 185 using sutures or other fasteners. In one such example, a plurality of sutures may be placed about the valve annulus 185, and then pushed or pulled thorough both the fabric covering 103 and the elongated core 102 by the operating physician. In some examples, the operating physician may place a suture in a plurality of locations distributed about an entire circumference (e.g., 360 degrees around) the annuloplasty ring 100. Additional details of how various sutures may be used secure annuloplasty rings to tissue are disclosed in U.S. Pat. No. 11,337,808, previously incorporated by reference above. Once the annuloplasty ring 100 has been implanted, it may reshape or constrict the valve annulus 185 to cause the leaflets of the valve to coapt, thereby treating an enlarged or diseased state of the valve 186.

In some examples, after the surgical implantation of the annuloplasty ring 100, the second regions 110 may, by virtue of the first stiffener 112, the second stiffener 114, or additional stiffeners, resist stretching of both posterior and anterior areas of the valve annulus 185 upon each beat of the patient's heart. Additionally, after the surgical implantation of the annuloplasty ring 100, the patient may, as is well known in the art, be monitored periodically to determine whether the valve 186 will relapse into a diseased state where regurgitant flow is detected.

Finally, if the valve 186 deteriorates to a point where complete replacement becomes necessary, a prosthetic valve may be inserted into the aperture 106 of the annuloplasty ring 100 during a second surgical or transcatheter implantation procedure. For example, a prosthetic valve may be guided into the annuloplasty ring 100 using various tools, devices, and surgical approaches known in the art. Additionally, as the prosthetic valve enters the aperture 106 of the annuloplasty ring 100, the elongated core 102 may elastically stretch and/or deform to closely conform to the circular geometry of the prosthetic valve, such as shown in FIG. 7, and thereby prevent subsequent paravalvular leakage by continuously applying a significant inward radial force about the entire circumference of the prosthetic valve.

Moreover, as the annuloplasty ring 100 remains sutured to tissue of the valve annulus 185 during such a procedure, the circular deformation of the annuloplasty ring 100 caused by the insertion of a prosthetic valve thereinto will cause a corresponding change in shape of the valve annulus 185. Additional details of how a prosthetic valve may be positioned within a previously implanted annuloplasty ring are disclosed in U.S. Pat. No. 11,337,808, previously incorporated by reference above.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. An annuloplasty ring for receiving a replacement heart valve, the annuloplasty ring comprising:
an elongated core including a first region and a second region, wherein the first region is more stretchable than the second region and is adapted for positioning at commissures of a target native heart valve, the first region being made solely from an elastic material;
a cover encompassing the elongated core;
wherein the elongated core is stretchable from a resting state to a stretched state to receive the replacement heart valve; and
wherein the first region is adapted to impart a squeezing force on the replacement heart valve so as to prevent paravalvular leakage.

2. The annuloplasty ring of claim 1, wherein the second region includes a first stiffener adapted to help maintain a shape of a posterior area of the annuloplasty ring.

3. The annuloplasty ring of claim 2, wherein the first stiffener comprises a wire including a pair of end loops.

4. The annuloplasty ring of claim 3, wherein the first stiffener extends between about 110 degrees and about 130 degrees around an aperture of the annuloplasty ring.

5. The annuloplasty ring of claim 3, wherein the second region includes a second stiffener adapted to help maintain a shape of an anterior area of the annuloplasty ring.

6. The annuloplasty ring of claim 5, wherein the first stiffener or the second stiffener defines a vertical curvature to cause the annuloplasty ring to form a three-dimensional saddle shape.

7. The annuloplasty ring of claim 6, wherein the first stiffener defines a length that is longer than a length defined by the second stiffener.

8. The annuloplasty ring of claim 1, wherein the elongated core forms a complete loop.

9. An annuloplasty ring for receiving a replacement heart valve, the annuloplasty ring comprising:
an elongated core comprising a pair of first regions and a pair of second regions, wherein the pair of first regions are adapted for positioning at commissures of a target native heart valve are more stretchable than the pair of second regions, and wherein the pair of first regions are comprised solely of a silicone-based material;
a cover encompassing the elongated core;
wherein the elongated core is stretchable from a resting state to a stretched state to receive the replacement heart valve; and
wherein the pair of first segments are configured to impart a squeezing force to the replacement heart valve so as to prevent paravalvular leakage.

10. The annuloplasty ring of claim 9, wherein the pair of second regions are separated from each other by the pair of first regions.

11. The annuloplasty ring of claim 10, wherein the pair of second regions and the pair of first regions collectively form a complete loop.

12. The annuloplasty ring of claim 10, wherein one of the pair of second regions includes a first stiffener adapted to help maintain a shape of a posterior area of the annuloplasty ring; and wherein another of the pair of second regions includes a second stiffener adapted to help maintain the shape of an anterior area of the annuloplasty ring.

13. The annuloplasty ring of claim 12, wherein the first stiffener and the second stiffener are curved to cause the annuloplasty ring to form a three-dimensional saddle shape.

14. The annuloplasty ring of claim 13, wherein the first stiffener and the second stiffener are each comprised of a polished wire defining a pair of end loops.

15. The annuloplasty ring of claim 14, wherein a ratio between a maximum width of an aperture defined by the elongated core and a maximum length of the aperture is about 4:3, and wherein the maximum width is measured between the pair of second regions the maximum length is measured between the pair of first regions.

16. A method of treating a diseased heart valve, the method comprising:
implanting an annuloplasty ring around a heart valve to cause leaflets of the heart valve to coapt, wherein the annuloplasty ring comprises a pair of first regions made solely from a silicone-based material, and wherein pair of first regions are more stretchable than the pair of second regions to allow the annuloplasty ring to be stretched from a resting state to a stretched state upon each opening of the heart valve;
monitoring the heart valve periodically for an extended period; and
installing a prosthetic valve within said annuloplasty ring if the heart valve relapses into a diseased state.

17. The method of claim 16, wherein implanting the annuloplasty ring around the diseased heart valve includes positioning each of the pair of first regions at a commissure of the heart valve where the pair of leaflets meet.

18. The method of claim 17, wherein implanting the annuloplasty ring around the heart valve includes positioning each of the pair of second regions along a leaflet of the heart valve.

19. The method of claim 18, wherein positioning each of the pair of second regions along a leaflet of the heart valve includes:
resisting, by a first stiffener of the pair of second regions, stretching of a posterior area of the annuloplasty ring; and
resisting, by a second stiffener of the pair of second regions, stretching of an anterior area of the annuloplasty ring.

20. The method of claim 16, wherein the method first comprises selecting the annuloplasty ring from a plurality of annuloplasty rings based on a maximum width of an aperture of the annuloplasty ring and a maximum length, and wherein a ratio between the maximum width and the maximum length is about 4:3.
